(19) United States
(12) Patent Application Publication
(10) Pub. No.: US 2024/0327379 A1
(43) Pub. Date: Oct. 3, 2024

(54) MANDELATE FORM OF 1-(4-(((6-AMINO-5-(4-PHENOXYPHENYL)PYRIMIDIN-4-YL)AMINO)METHYL)PIPERIDIN-1-YL)PROP-2-EN-1-ONE

(71) Applicant: Sandoz AG, Basel (CH)

(72) Inventors: Hannes Lengauer, Kundl (AT); Renate Margreiter, Kundl (AT); Arthur Pichler, Kundl (AT)

(73) Assignee: Sandoz AG, Basel (CH)

(21) Appl. No.: 18/578,864

(22) PCT Filed: Jul. 15, 2022
(86) PCT No.: PCT/EP2022/069956

§ 371 Date: Jan. 12, 2024

(30) Foreign Application Priority Data
Jul. 15, 2021 (EP) 21185753.7

(51) Int. Cl. C07D 401/12; A61K 31/506; C07C 59/50

(52) U.S. Cl. CPC C07D 401/12; A61K 31/506; C07C 59/50

(57) ABSTRACT

The present invention relates to a mandelate form of 1-(4-(((6-amino-5-(4-phenoxyphenyl) pyrimidin-4-yl)amino)methyl)piperidin-1-yl)prop-2-en-1-one (INN: evobrutinib) and a process of producing the same. Furthermore, the invention relates to a pharmaceutical composition comprising the mandelate form of evobrutinib and at least one pharmaceutically acceptable excipient. The pharmaceutical composition of the present invention can be used as a medicament, in particular for the treatment and/or prevention of multiple sclerosis.

### FIELD OF THE INVENTION

The present invention relates to a mandelate form of 1-(4-(((6-amino-5-(4-phenoxyphenyl)pyrimidin-4-yl)amino)methyl)piperidin-1-yl)prop-2-en-1-one (INN: evobrutinib) and a process of producing the same. Furthermore, the invention relates to a pharmaceutical composition comprising the mandelate form of evobrutinib and at least one pharmaceutically acceptable excipient. The pharmaceutical composition of the present invention can be used as a medicament, in particular for the treatment and/or prevention of multiple sclerosis.

### BACKGROUND OF THE INVENTION

Evobrutinib is an oral, highly selective inhibitor of Bruton's tyrosine kinase (BTK) which is important in the development and functioning of various immune cells including B-lymphocytes and macrophages. Evobrutinib is designed to inhibit primary B-cell responses such as proliferation and antibody and cytokine release, without directly affecting T-cells. BTK inhibition is thought to suppress autoantibody-producing cells, which preclinical research suggests may be therapeutically useful in certain autoimmune diseases. Evobrutinib is currently under clinical investigation as potential treatment of relapsing multiple sclerosis (RMS).

Chemically also designated 1-(4-(((6-amino-5-(4-phenoxyphenyl)pyrimidin-4-yl)amino)methyl)piperidin-1-yl)prop-2-en-1-one, evobrutinib can be represented by the chemical structure as depicted in Formula (A)

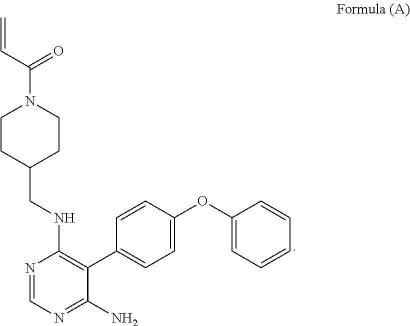 [SMILES: C=CC(=O)N1CCC(CNc2ncnc(N)c2-c2ccc(Oc3ccccc3)cc2)CC1]

WO 2012/170976 A1 discloses evobrutinib (compound A252) and a process for its preparation. WO 2018/154131 A1 discloses crystalline and amorphous forms of evobrutinib and various evobrutinib salt forms selected from the group consisting of malonate, succinate, oxalate, fumarate, maleate, malate and citrate.

Different solid-state forms of an active pharmaceutical ingredient (API) often possess different physical and chemical properties such as but not limited to dissolution rate, solubility, chemical stability, physical stability, hygroscopicity, melting point, morphology, flowability, bulk density and compressibility. Differences in physicochemical properties of solid-state forms can play a crucial role for the improvement of pharmaceutical compositions, for example, pharmaceutical formulations with improved dissolution profile and bioavailability or with improved stability or shelf-life can become accessible due to an improved solid-state form of an API. Also processing or handling of the API during the formulation process may be improved. New solid-state forms of an API can thus have desirable processing properties. They can be easier to handle, better suited for storage, and/or allow for better purification, compared to previously known solid-state forms.

It is thus an objective of the present invention to provide an improved solid-state form of evobrutinib.

### SUMMARY OF THE INVENTION

The inventors of the present invention have surprisingly found that when applying very specific crystallization conditions, a crystalline mandelate form of evobrutinib can be obtained.

The crystalline evobrutinib mandelate form of the present invention possesses one or more advantageous physicochemical properties selected from the group consisting of dissolution rate, solubility, chemical stability, physical stability, hygroscopicity, melting point, morphology, flowability, wettability, bulk density and compressibility.

In particular, the evobrutinib mandelate form of the present invention is highly crystalline (see FIG. 1 herein), shows good thermal stability and exhibits a sharp and well-defined melting point with a DSC onset temperature of about 118° C. (see Example 6 and FIG. 2 herein), possesses a low residual solvent content (see Example 7 and FIG. 3 herein) and is only slightly hygroscopic (see Example 8 and FIG. 4 herein).

### Abbreviations

PXRD powder X-ray diffractogram
DSC differential scanning calorimetry
TGA thermogravimetric analysis
GMS gravimetric moisture sorption
RT room temperature
RH relative humidity
API active pharmaceutical ingredient

### Definitions

As used herein the term “room temperature” refers to a temperature in the range of from 20 to 30° C.

The terms “evobrutinib mandelate”, “evobrutinib mandelate form” or “mandelate form of evobrutinib” which may be interchangeably used herein refer to a crystalline compound comprising about one molecule mandelic acid per molecule evobrutinib. The evobrutinib mandelate form of the present invention can be characterized by the chemical structure as depicted in Formula (B) hereinafter. Thereby, the interaction between evobrutinib free base and the mandelic acid molecules can be ionic (e.g. salt-like) and/or non-ionic (e.g. co-crystal like). The mandelic acid may exist as enantiomers. For example, mandelic acid may be present as S-enantiomer or as R-enantiomer. The evobrutinib mandelate form as disclosed herein includes both enantiomers, both as pure individual enantiomers, enriched enantiomers or non-specific mixtures of enantiomers.

The term “co-crystal” as used herein refers to crystalline materials composed of two or more different molecular and/or ionic compounds in the same crystal lattice that are associated by nonionic and noncovalent bonds, wherein at least two of the individual molecular and/or ionic compounds are solids at room temperature. Co-crystals are structurally readily distinguishable from salts because unlike salts, their components are in a neutral state and interact nonionically.

As used herein, the term “measured at a temperature in the range of from 20 to 30° C.” refers to a measurement under standard conditions. Typically, standard conditions mean a temperature in the range of from 20 to 30° C., i.e. at room temperature. Standard conditions can mean a temperature of about 22° C. Typically, standard conditions can additionally mean a measurement under 20-50% relative humidity.

The term “reflection” with regard to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material, whereas amorphous material is defined as solid material, which lacks long-range order and only displays short-range order, thus resulting in broad scattering. According to literature, long-range order e.g. extends over approximately 100 to 1000 atoms, whereas short-range order is over a few atoms only (see “Fundamentals of Powder Diffraction and Structural Characterization of Materials” by Vitalij K. Pecharsky and Peter Y. Zavalij, Kluwer Academic Publishers, 2003, page 3).

The term “essentially the same” with reference to powder X-ray diffraction means that variabilities in reflection positions and relative intensities of the reflections are to be taken into account. For example, a typical precision of the 2-Theta values is in the range of ±0.2° 2-Theta, preferably in the range of ±0.1° 2-Theta. Thus, a reflection that usually appears at 7.2° 2-Theta for example can appear between 7.0° and 7.4° 2-Theta, preferably between 7.1 and 7.3° 2-Theta on most X-ray diffractometers under standard conditions. Furthermore, one skilled in the art will appreciate that relative reflection intensities will show inter-apparatus variability as well as variability due to degree of crystallinity, preferred orientation, sample preparation and other factors known to those skilled in the art and should be taken as qualitative measure only.

The term “solid-state form” as used herein refers to any crystalline and/or amorphous phase of a compound. Crystalline phases include anhydrous/non-solvated forms of a compound and their polymorphs, hydrates and solvates of a compound and their polymorphs, salts and co-crystals of a compound and their polymorphs and pseudopolymorphic forms and any mixtures thereof.

As used herein, the term “essentially free of any other solid-state form” with reference to the composition comprising the evobrutinib mandelate form of the present invention, means that the evobrutinib mandelate form contains at most 20 weight %, preferably at most 10 weight %, more preferably at most 5 weight %, 4 weight %, 3 weight %, 2 weight % or 1 weight % of any other solid-state form of evobrutinib, based on the total weight of the composition.

The mandelate form of evobrutinib of the present invention may be referred to herein as being characterized by a powder X-ray diffractogram “as shown in” a figure. The person skilled in the art understands that factors such as variations in instrument type, response and variations in sample directionality, sample concentration, sample purity, sample history and sample preparation may lead to variations, for example relating to the exact reflection positions and intensities. However, a comparison of the graphical data in the figure herein with the graphical data generated for an unknown physical form and the confirmation that two sets of graphical data relate to the same crystal form is well within the knowledge of a person skilled in the art.

As used herein, the term “mother liquor” refers to the solution remaining after crystallization of a solid from said solution.

As used herein, the term “about” means within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

The term “pharmaceutically acceptable excipient” as used herein refers to substances, which do not show a significant pharmacological activity at the given dose and that are added to a pharmaceutical composition in addition to the active pharmaceutical ingredient. Excipients may take the function of vehicle, diluent, release agent, disintegrating agent, dissolution modifying agent, absorption enhancer, wetting agent, stabilizer or a manufacturing aid among others.

### BRIEF DESCRIPTION OF THE FIGURES

FIG. 1: illustrates a representative PXRD of the evobrutinib mandelate form according to the present invention. The x-axis shows the scattering angle in ° 2-Theta, the y-axis shows the intensity of the scattered X-ray beam in counts of detected photons.

FIG. 2: illustrates a representative DSC curve of the evobrutinib mandelate form according to the present invention. The x-axis shows the temperature in degree Celsius (C), the y-axis shows the heat flow rate in Watt per gram (W/g) with endothermic peaks going up.

FIG. 3: illustrates a representative TGA curve of the evobrutinib mandelate form of the present invention. The x-axis shows the temperature in degree Celsius (° C.), the y-axis shows the mass (loss) of the sample in weight percent (w-%).

FIG. 4: illustrates representative GMS isotherms of the evobrutinib mandelate form of the present invention in the range of from 0 to 90% RH. The x-axis displays the relative humidity in percent (%) measured at a temperature of (25.0±0.1)° C., the y-axis displays the equilibrium mass change in weight percent (w-%). Sample weight at 0% RH at the end of the desorption curve is used as reference weight. Sorption curve points are displayed as triangles, desorption curve points as squares.

### DETAILED DESCRIPTION OF THE INVENTION

The present invention provides a crystalline mandelate form of evobrutinib.

In a first aspect, the invention relates to a crystalline evobrutinib mandelate form characterized by having the chemical structure as depicted in Formula (B)

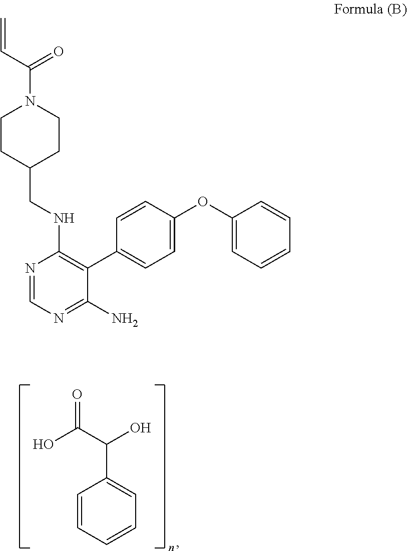

wherein n is in the range of from 0.8 to 1.2, preferably of from 0.9 to 1.1, even more preferably of from 0.95 to 1.05 and most preferably n is 1.0.

In one embodiment, the invention relates to a crystalline evobrutinib mandelate form characterized by having the chemical structure as depicted in Formula (C)

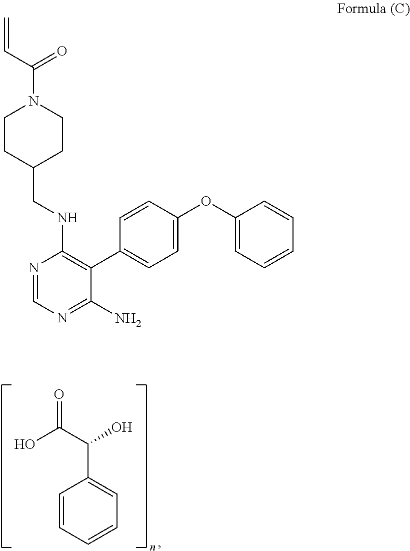

wherein n is in the range of from 0.8 to 1.2, preferably of from 0.9 to 1.1, even more preferably of from 0.95 to 1.05 and most preferably n is 1.0.

In a preferred embodiment, the invention relates to a crystalline evobrutinib mandelate form characterized by having the chemical structure as depicted in Formula (D)

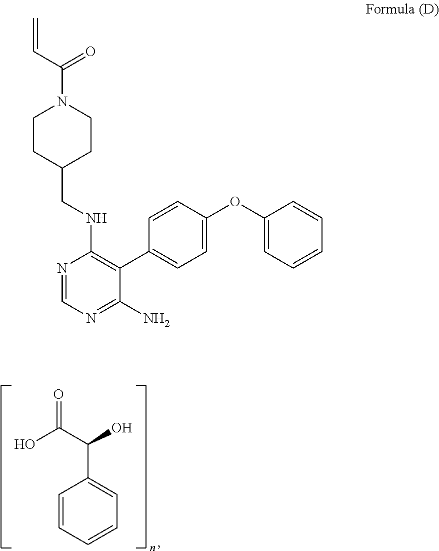

wherein n is in the range of from 0.8 to 1.2, preferably of from 0.9 to 1.1, even more preferably of from 0.95 to 1.05 and most preferably n is 1.0.

In another embodiment, the invention relates to a crystalline evobrutinib mandelate form characterized in that the crystalline evobrutinib mandelate form is present as acid addition salt, in particular as crystalline evobrutinib mandelate salt.

In a particular preferred embodiment, the invention relates to a crystalline evobrutinib mandelate form characterized in that the crystalline evobrutinib mandelate form is present as a co-crystal comprising evobrutinib as active pharmaceutical ingredient and mandelic acid as co-crystal former in the same crystal lattice, wherein the interaction between evobrutinib and mandelic acid is of nonionic and noncovalent nature.

In an alternative embodiment, the invention relates to a crystalline evobrutinib mandelate form characterized in that the crystalline evobrutinib mandelate form is present as a co-crystal and acid addition salt mixture.

The crystalline evobrutinib mandelate form of the present invention may be characterized by analytical methods well known in the field of the pharmaceutical industry for characterizing crystalline solids. Such methods comprise but are not limited to powder X-ray diffraction, differential scanning calorimetry, thermogravimetric analysis and gravimetric moisture sorption. The evobrutinib mandelate form of the present invention may be characterized by one of the aforementioned analytical methods or by combining two or more of them. In particular, the mandelate form of evobrutinib of the present invention may be characterized by any one of the following embodiments or by combining two or more of the following embodiments.

In one embodiment the invention relates to a crystalline evobrutinib mandelate form characterized by having a PXRD comprising reflections at 2-Theta angles of:
(7.2±0.2)°, (8.3±0.2)° and (18.3±0.2)°; or
(7.2±0.2)°, (8.3±0.2)°, (15.6±0.2)° and (18.3±0.2)°; or
(7.2±0.2)°, (8.3±0.2)°, (13.8±0.2)°, (15.6±0.2)° and (18.3±0.2)°; or
(7.2±0.2)°, (8.3±0.2)°, (11.9±0.2)°, (13.8±0.2)°, (15.6±0.2)° and (18.3±0.2)°; or
(7.2±0.2)°, (8.3±0.2)°, (11.9±0.2)°, (12.5±0.2)°, (13.8±0.2)°, (15.6±0.2)° and (18.3±0.2)°; or
(7.2±0.2)°, (8.3±0.2)°, (11.9±0.2)°, (12.5±0.2)°, (13.9±0.2)°, (15.6±0.2)°, (18.3±0.2)° and (22.1±0.2)°; or
(7.2±0.2)°, (8.3±0.2)°, (9.1±0.2)°, (11.9±0.2)°, (12.5±0.2)°, (13.8±0.2)°, (15.6±0.2)°, (18.3±0.2)° and (22.1±0.2)°; or
(7.2±0.2)°, (8.3±0.2)°, (9.1±0.2)°, (10.7±0.2)°, (11.9±0.2)°, (12.5±0.2)°, (13.8±0.2)°, (15.6±0.2)°, (18.3±0.2)° and (22.1±0.2)°; or
(7.2±0.2)°, (8.3±0.2)°, (9.1±0.2)°, (10.7±0.2)°, (11.9±0.2)°, (12.5±0.2)°, (13.8±0.2)°, (15.6±0.2)°, (16.7±0.2)°, (18.3±0.2)° and (22.1±0.2)°; or
(7.2±0.2)°, (8.3±0.2)°, (9.1±0.2)°, (10.7±0.2)°, (11.9±0.2)°, (12.5±0.2)°, (13.8±0.2)°, (15.6±0.2)°, (16.7±0.2)°, (18.3±0.2)°, (18.9±0.2)° and (22.1±0.2)°;
when measured at a temperature in the range of from 20 to 30° C. with Cu-Kalpha_{1,2} radiation having a wavelength of 0.15419 nm.

In a further embodiment the invention relates to a crystalline evobrutinib mandelate form characterized by having a PXRD comprising reflections at 2-Theta angles of:
(7.2±0.1)°, (8.3±0.1)° and (18.3±0.1)°; or
(7.2±0.1)°, (8.3±0.1)°, (15.6±0.1)° and (18.3±0.1)°; or
(7.2±0.1)°, (8.3±0.1)°, (13.8±0.1)°, (15.6±0.1)° and (18.3±0.1)°; or
(7.2±0.1)°, (8.3±0.1)°, (11.9±0.1)°, (13.8±0.1)°, (15.6±0.1)° and (18.3±0.1)°; or
(7.2±0.1)°, (8.3±0.1)°, (11.9±0.1)°, (12.5±0.1)°, (13.8±0.1)°, (15.6±0.1)° and (18.3±0.1)°; or
(7.2±0.1)°, (8.3±0.1)°, (11.9±0.1)°, (12.5±0.1)°, (13.9±0.1)°, (15.6±0.1)°, (18.3±0.1)° and (22.1±0.1)°; or
(7.2±0.1)°, (8.3±0.1)°, (9.1±0.1)°, (11.9±0.1)°, (12.5±0.1)°, (13.8±0.1)°, (15.6±0.1)°, (18.3±0.1)° and (22.1±0.1)°; or
(7.2±0.1)°, (8.3±0.1)°, (9.1±0.1)°, (10.7±0.1)°, (11.9±0.1)°, (12.5±0.1)°, (13.8±0.1)°, (15.6±0.1)°, (18.3±0.1)° and (22.1±0.1)°; or
(7.2±0.1)°, (8.3±0.1)°, (9.1±0.1)°, (10.7±0.1)°, (11.9±0.1)°, (12.5±0.1)°, (13.8±0.1)°, (15.6±0.1)°, (16.7±0.1)°, (18.3±0.1)° and (22.1±0.1)°; or
(7.2±0.1)°, (8.3±0.1)°, (9.1±0.1)°, (10.7±0.1)°, (11.9±0.1)°, (12.5±0.1)°, (13.8±0.1)°, (15.6±0.1)°, (16.7±0.1)°, (18.3±0.1)°, (18.9±0.1)° and (22.1±0.1)°;
when measured at a temperature in the range of from 20 to 30° C. with Cu-Kalpha_{1,2} radiation having a wavelength of 0.15419 nm.

In another embodiment the present invention relates to a crystalline evobrutinib mandelate form characterized by having a PXRD comprising reflections at 2-Theta angles of (7.2±0.2)°, (8.3±0.2)°, (11.9±0.2)°, (12.5±0.2)°, (13.8±0.2)°, (15.6±0.2)°, (18.3±0.2)°, (18.9±0.2)°, (22.1±0.2)° and (24.0±0.2)°, when measured at a temperature in the range of from 20 to 30° C. with Cu-Kalpha_{1,2} radiation having a wavelength of 0.15419 nm.

In an additional embodiment the invention relates to a crystalline evobrutinib mandelate form characterized by having a PXRD comprising reflections at 2-Theta angles of (7.2±0.1)°, (8.3±0.1)°, (11.9±0.1)°, (12.5±0.1)°, (13.8±0.1)°, (15.6±0.1)°, (18.3±0.1)°, (18.9±0.1)°, (22.1±0.1)° and (24.0±0.1)°, when measured at a temperature in the range of from 20 to 30° C. with Cu-Kalpha_{1,2} radiation having a wavelength of 0.15419 nm.

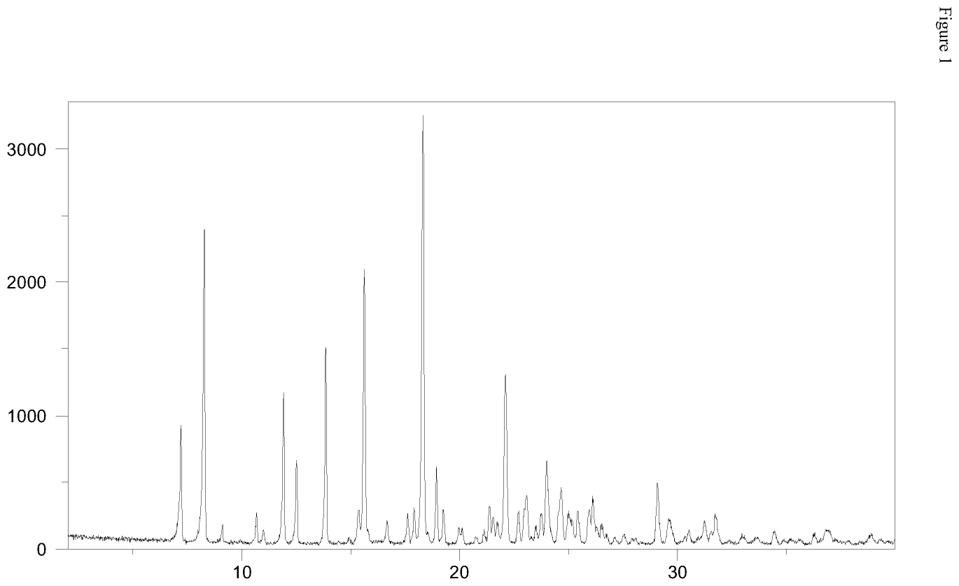

In another embodiment the invention relates to a crystalline evobrutinib mandelate form characterized by having a PXRD essentially the same as shown in FIG. 1 of the present invention, when measured at a temperature in the range of from 20 to 30° C. with Cu-Kalpha_{1,2} radiation having a wavelength of 0.15419 nm.

In yet another embodiment, the present invention relates to a crystalline evobrutinb mandelate form characterized by having a DSC curve comprising an endothermic peak, preferably a single endothermic peak having an onset at a temperature of (118±5)° C., preferably of (118±3)° C., more preferably of (118±2)° C. and most preferably of (118±1)° C., when measured at a heating rate of 10 K/min.

In a further embodiment, the present invention relates to a crystalline evobrutinib mandelate form characterized by having a DSC curve comprising an endothermic peak, preferably a single endothermic peak having a maximum at a temperature of (122±5)° C., preferably of (122±3)° C., more preferably of (122±2)° C. and most preferably of (122±1)° C., when measured at a heating rate of 10 K/min.

In another embodiment, the present invention relates to a crystalline evobrutinib mandelate form characterized by having a TGA curve showing a mass loss of not more than 0.5 w-%, preferably of not more than 0.4 w-%, 0.3 w-%, 0.2 w-% or 0.1 w-% based on the weight of the crystalline form, when heated from 25 to 170° C. at a rate of 10 K/min.

In a further embodiment, the present invention relates to a crystalline evobrutinib mandelate form characterized by showing a mass change of not more than 1.0 w-%, preferably of not more than 0.6 w-%, based on the weight of the crystalline form, when measured with GMS at a relative humidity in the range of from 0 to 90% and a temperature of (25.0±1.0)° C.

In one embodiment, the present invention relates to a crystalline evobrutinib mandelate form characterized in being anhydrous.

In another embodiment, the present invention relates to a crystalline evobrutinib mandelate form characterized in being non-solvated.

In further embodiment, the present invention relates to a crystalline evobrutinib mandelate form characterized in being slightly hygroscopic.

In another aspect, the present invention relates to a composition comprising the crystalline evobrutinib mandelate form of the present invention as defined in any one of the above described embodiments, said composition being essentially free of any other solid-state form of evobrutinib. For example, a composition comprising the evobrutinib mandelate form of the present invention comprises at most 20 weight %, preferably at most 10 weight %, more preferably at most 5 weight %, 4 weight %, 3 weight %, 2 weight % or 1 weight % of any other solid-state form of evobrutinib, based on the total weight of the composition.

In a further aspect the present invention relates to the use of the evobrutinib mandelate form of the present invention or the composition comprising the evobrutinib mandelate form as defined in any one of the above described aspects and their corresponding embodiments for the preparation of a pharmaceutical composition.

In a further aspect, the present invention relates to a pharmaceutical composition comprising the evobrutinib mandelate form of the present invention or the composition comprising the evobrutinib mandelate form as defined in any one of the above described aspects and their corresponding embodiments, preferably in a predetermined and/or effective amount, and at least one pharmaceutically acceptable excipient.

Preferably, the predetermined and/or effective amount of the evobrutinib mandelate form of the present invention is in the range of from 20 to 300 mg calculated as evobrutinib free base. For example the predetermined and/or effective amount of the evobrutinib mandelate form of the present invention is selected from the group consisting of 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg 275 mg, 280 mg, 285 mg, 290 mg, 295 mg and 300 mg calculated as evobrutinib free base. In a particular preferred embodiment the predetermined and/or effective amount of the evobrutinib mandelate form of the present invention is selected from the group consisting of 25 mg, 45 mg and 75 mg, calculated as evobrutinib free base.

Preferably, the pharmaceutical composition of the present invention as described above is an oral solid dosage form. In a preferred embodiment the oral solid dosage form is a tablet or a capsule. In a particularly preferred embodiment the pharmaceutical composition of the present invention is a tablet, preferably a film-coated tablet comprising a tablet core and a coating.

The tablet or tablet core may be prepared by mixing the evobrutinib mandelate form of the present invention with at least one pharmaceutically acceptable excipient followed by compressing the mixture. Optionally, a granulation step such as a wet or dry granulation step is performed before compression. Preferably, the tablet core is subsequently coated with a film-coat. Methods of preparing such tablets, tablet cores and film-coated tablets are well known in the pharmaceutical arts.

In a further aspect, the present invention relates to the evobrutinib mandelate form, the composition comprising the evobrutinib mandelate form or the pharmaceutical composition comprising the evobrutinib mandelate form or the composition comprising the evobrutinib mandelate form as defined in any one of the above described aspects and their corresponding embodiments for use as a medicament.

In yet another aspect, the present invention relates to the evobrutinib mandelate form, the composition comprising the evobrutinib mandelate form or the pharmaceutical composition comprising the evobrutinib mandelate form or the composition comprising the evobrutinib mandelate form as defined in any one of the above described aspects and their corresponding embodiments for use in the treatment or prevention of a condition selected from the group consisting of multiple sclerosis e.g. relapsing multiple sclerosis.

In an alternative embodiment, the invention concerns a method of treating or preventing multiple sclerosis, e.g. relapsing multiple sclerosis said method comprising administering an effective amount of the evobrutinib mandelate form, the composition comprising the evobrutinib mandelate form or the pharmaceutical composition comprising the evobrutinib mandelate form or the composition comprising the evobrutinib mandelate form as defined in any one of the above described aspects and their corresponding embodiments to a patient in need of such a treatment.

### EXAMPLES

The following non-limiting examples are illustrative for the disclosure and are not to be construed as to be in any way limiting for the scope of the invention.

### Example 1: Preparation of Evobrutinib Mandelate Seed Crystals

Amorphous evobrutinib (50 mg, e.g. prepared according to Example 4 hereinafter) and mandelic acid (19.7 mg, 1.1 eq) were suspended in a mixture of ethanol (0.1 mL) and diethyl ether (0.4 ml) at room temperature for 4 hours and the crystals were collected by centrifugation to yield the crystalline evobrutinib mandelate form of the present invention.

### Example 2: Preparation of the Evobrutinib Mandelate Form of the Present Invention

Amorphous evobrutinib (500 mg, e.g. prepared according to Example 4 hereinafter) and mandelic acid (186.1 mg, 1.1 eq) were dissolved in ethanol (5 mL) at room temperature. To the clear solution diethyl ether (10 mL) and seed crystals (e.g. prepared according to Example 1 herein) were added and the mixture was stirred at room temperature overnight. The obtained crystals were collected by filtration and dried under vacuum (˜5 mbar) at room temperature to yield the crystalline evobrutinib mandelate form of the present invention

Yield: 348 mg

### Example 3: Preparation of the Evobrutinib Mandelate Form of the Present Invention

Crystalline evobrutinib (505 mg, e.g. prepared according to Example 1 of WO 2018/154131 A1) and mandelic acid (203.1 mg, 1.1 eq) were dissolved in ethanol (5 mL) at room temperature. To the clear solution diethyl ether (10 mL) and seed crystals (e.g. prepared according to Example 1 herein) were added and the mixture was stirred at room temperature overnight. The obtained crystals were collected by filtration and dried under vacuum (˜5 mbar) at room temperature to yield the evobrutinib mandelate form of the present invention

Yield: 374 mg

### Example 4: Preparation of Amorphous Evobrutinib

Crystalline evobrutinib (10.017 g, e.g. prepared according to the procedure disclosed in WO 2012/170976 A1) was dissolved in aqueous acetonitrile (1000 mL, 75% v/v). The solution was lyophilized to yield amorphous evobrutinib quantitatively.

### Example 5: Powder X-Ray Diffraction

PXRD was performed with a PANalytical X'Pert PRO diffractometer equipped with a theta/theta coupled goniometer in transmission geometry, Cu-Kalpha_{1,2} radiation (wavelength 0.15419 nm) with a focusing mirror and a solid state PIXcel detector. The diffractogram was recorded at a tube voltage of 45 kV and a tube current of 40 mA, applying a step size of 0.013° 2-theta with 40 s per step (255 channels) in the angular range of 2° to 40° 2-Theta at ambient conditions. A typical precision of the 2-theta values is in the range of ±0.2° 2-Theta, preferably of 0.1° 2-Theta.

A representative diffractogram of the crystalline evobrutinib mandelate form of the present invention is displayed in FIG. 1 hereinafter. The corresponding reflection list is provided in Table 1 below.

TABLE 1
Reflection (peak) positions of the evobrutinib mandelate form
according to the present invention in the range of from 2 to
30° 2-Theta; A typical precision of the 2-Theta values
is in the range of ±0.2° 2-Theta, preferably of ±0.1° 2-Theta.
Reflection position
[° 2-Theta]

7.2
8.3
9.1
10.7
11.0
11.9
12.5
13.8
15.4
15.6
16.7
17.6
17.9
18.3
18.9
19.2
20.0
20.1
21.1
21.4
21.5
21.7
22.1
22.7
23.1
23.5
23.7
24.0
24.6
25.0
25.4
25.9
26.1
26.5
29.1
29.6

### Example 6: Differential Scanning Calorimetry (DSC)

DSC was performed on a Mettler Polymer DSC R instrument. The sample (3.08 mg) was heated in a 40 microliter aluminium pan with a pierced aluminium lid from 25 to 250° C. at a rate of 10° K/min. Nitrogen (purge rate 50 mL/min) was used as purge gas.

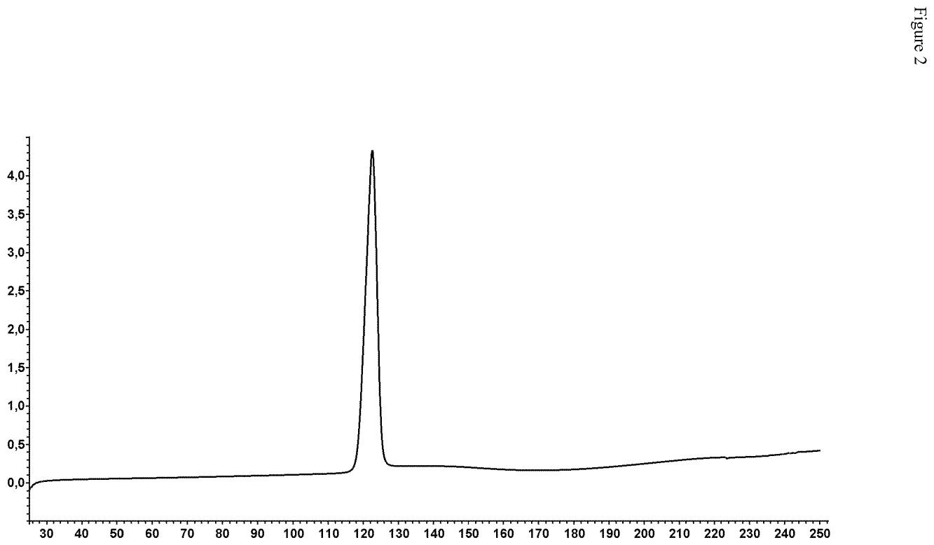

A representative DSC curve of the crystalline evobrutinib mandelate form of the present invention is shown in FIG. 2 hereinafter and shows a single endothermic peak with an onset temperature of about 118° C. and a peak temperature of about 122° C., which is due to melting.

### Example 7: Thermogravimetric Analysis

TGA was performed on a Mettler TGA/DSC 1 instrument. The sample (6.53 mg) was heated in a 100 microliter aluminium pan, closed with an aluminium lid, from 25 to 250° C. at a rate of 10 K/min. The lid was automatically pierced at the beginning of the measurement. Nitrogen (purge rate 30 mL/min) was used as purge gas.

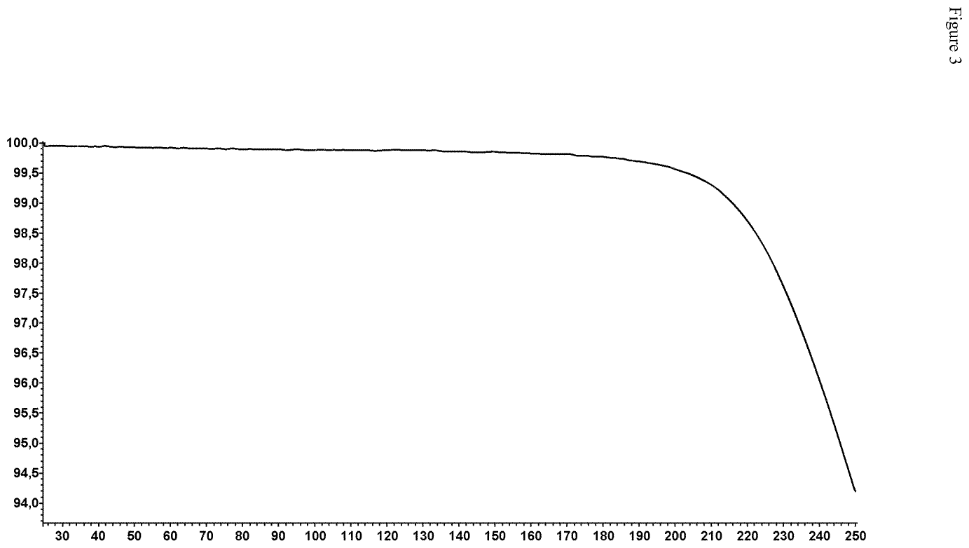

A representative TGA curve of the crystalline evobrutinib mandelate form of the present invention is displayed in FIG. 3 hereinafter and shows no significant mass loss until the sample melts. For example, the mass loss from the beginning of the measurement until about 170° C. was only about 0.1%. Therefore, the evobrutinib mandelate form of the present invention can be assigned an anhydrous and non-solvated crystal form.

### Example 8: Gravimetric Moisture Sorption

Gravimetric moisture sorption was performed with an SPSx-1μ moisture sorption analyzer (ProUmid, Ulm). The measurement cycle was started at ambient relative humidity (RH) of 30%. RH was then decreased to 5% in 5% steps, followed by a further decrease to 3% and to 0%. Afterwards RH was increased from 0% to 90% in a sorption cycle and subsequently decreased to 0% in a desorption cycle each in 5% steps. Finally, RH was increased to ambient relative humidity of 30% in 5% steps. The time per step was set to a minimum of 2 hours and a maximum of 6 hours. If an equilibrium condition with a constant mass of =0.01% within 1 hour was reached before the maximum time for all examined samples the sequential humidity step was applied before the maximum time of 6 hours. If no equilibrium was achieved the consecutive humidity step was applied after the maximum time of 6 hours. The temperature was (25±0.1)° C.

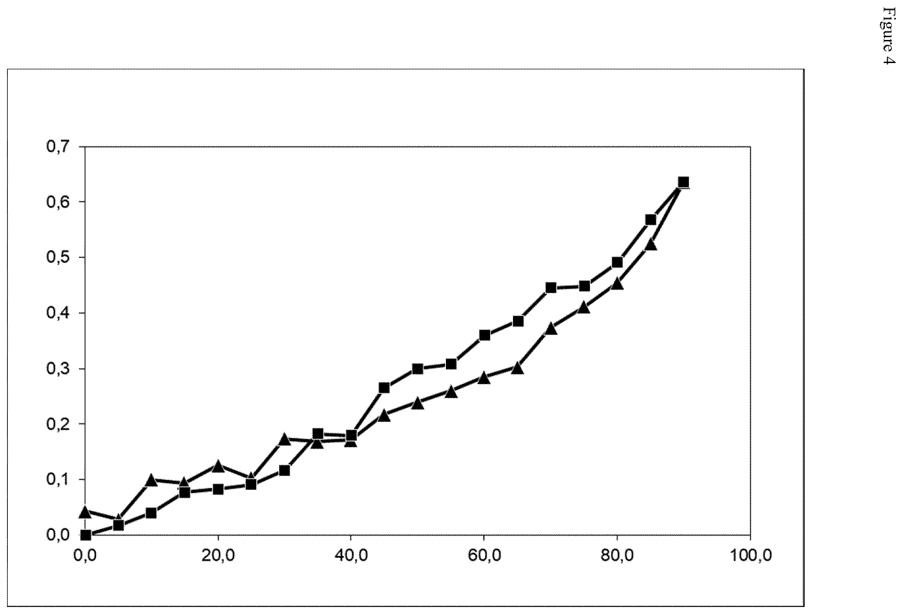

The moisture sorption/desorption isotherms of the crystalline evobrutinib mandelate form of the present invention in the range from 0-90% RH are displayed in FIG. 4. The mass difference in the sorption cycle between 0 and 80% RH is only about 0.5 w-% and the maximum uptake between 0 and 90% RH is only about 0.6% indicating the slightly hygroscopic behaviour of this crystalline form. The lack of a significant hysteresis between the sorption and the desorption isotherms indicates that no structural changes appear during the experiment. This assumption is strengthened by the fact that the sample still shows the same PXRD after the experiment.

1. A crystalline 1-(4-(((6-amino-5-(4-phenoxyphenyl)pyrimidin-4-yl)amino)methyl)piperidin-1-yl)prop-2-en-1-one (evobrutinib) mandelate form having a chemical structure as depicted in Formula (B) 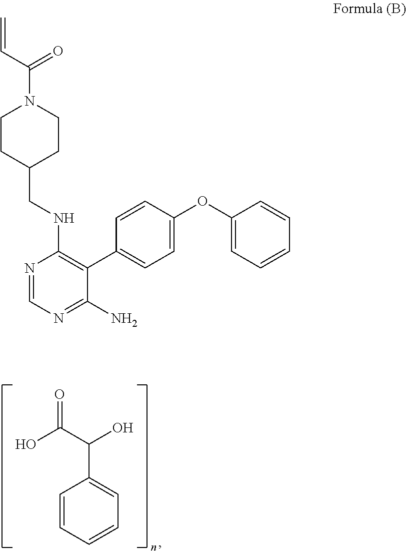
wherein n is is in the range of from 0.8 to 1.2, characterized by having a powder X-ray diffractogram comprising reflections at 2-Theta angles of (7.2±0.2)°, (8.3±0.2)° and (18.3±0.2)°, when measured at a temperature in the range of from 20 to 30° C. with Cu-Kalpha_{1,2} radiation having a wavelength of 0.15419 nm.

2. The crystalline evobrutinib mandelate form according to claim 1 characterized by having a powder X-ray diffractogram comprising additional reflections at 2-Theta angles of (13.8±0.2)° and/or (15.6±0.2)°, when measured at a temperature in the range of from 20 to 30° C. with Cu-Kalpha_{1,2} radiation having a wavelength of 0.15419 nm.

3. The crystalline evobrutinib mandelate form according to claim 1 characterized by having a differential scanning calorimetry curve comprising an endothermic peak having an onset at a temperature of (118±5)° C., when measured at a heating rate of 10 K/min.

4. The crystalline evobrutinib mandelate form according to claim 1 characterized by having a thermogravimetric analysis curve showing a mass loss of not more than 0.5 w-% based on the weight of the crystalline form, when heated from 25 to 170° C. at a rate of 10 K/min.

5. The crystalline evobrutinib mandelate form according to claim 1 characterized by showing a mass change of not more than 1.0 w based on the weight of the crystalline form, when measured with gravimetric moisture sorption at a relative humidity in the range of from 0 to 90% and a temperature of (25.0±1.0)° C.

6. The crystalline evobrutinib mandelate form according to claim 1 characterized in being anhydrous.

7. The crystalline evobrutinib mandelate form according claim 1 characterized in being non-solvated.

8. A composition comprising the crystalline evobrutinib mandelate form as defined in claim 1 characterized by comprising at most 20 weight % of any other solid-state form of evobrutinib, based on the total weight of the composition.

9. Process for preparing a pharmaceutical composition comprising the steps of providing the crystalline evobrutinib mandelate form as defined in claim 1; providing at least one pharmaceutically acceptable excipient; and obtaining said pharmaceutical composition.

10. A pharmaceutical composition comprising an effective and/or predetermined amount of the crystalline evobrutinib mandelate form as defined in claim 1 and at least one pharmaceutically acceptable excipient.

11. The pharmaceutical composition of claim 10, wherein the effective and/or predetermined amount of the crystalline evobrutinib mandelate form is selected from the group consisting of 25 mg, 45 mg and 75 mg, calculated as evobrutinib free base.

12. The pharmaceutical composition according to claim 10, wherein the pharmaceutical composition is an oral solid dosage form.

13. The pharmaceutical composition of claim 12, wherein the oral solid dosage form is a tablet or a capsule.

14. A method of treatment of a disease in a patient, comprising administering to the patient in need of such a treatment a crystalline evobrutinib mandelate form as defined in claim 1.

15. A method for the treatment and/or prophylaxis of multiple sclerosis, comprising providing the crystalline evobrutinib mandelate form as defined in claim 1, and administering said crystalline evobrutinib, said composition or said pharmaceutical composition to a patient in need of in the treatment and/or prophylaxis of multiple sclerosis.
